(19) United States
(12) Patent Application Publication
(10) Pub. No.: US 2006/0188491 A1
(43) Pub. Date: Aug. 24, 2006

(54) Use of nuclear material to therapeutically reprogram differentiated cells

(75) Inventors: Chauncey B. Sayre, Irvine, CA (US); Francisco J. Silva, Rancho Cucamonga, CA (US)

Correspondence Address: PRESTON GATES & ELLIS LLP, 1900 MAIN STREET, SUITE 600, IRVINE, CA, 92614-7319, US

(73) Assignee: PrimeGen Biotech, LLC, Irvine, CA (US)

(21) Appl. No.: 11/346,919

(22) Filed: Feb. 2, 2006

Related U.S. Application Data
(63) Continuation-in-part of application No. 11/060,131, filed on Feb. 16, 2005.
(60) Provisional application No. 60/649,847, filed on Feb. 2, 2005.
(60) Provisional application No. 60/588,146, filed on Jul. 15, 2004.

(51) Int. Cl. A61K 48/00; C12N 5/08; A61K 38/17

(52) U.S. Cl. 424/93.21; 435/366; 514/12

(57) ABSTRACT

Methods are provided for therapeutically reprogramming differentiated cells with nuclear extracts from pluripotent stem cells to generate a pluripotent epigenetic state in the differentiated cells. Additionally, therapeutically programmed cells for therapeutic use in patients are provided. Therapeutically programmed cells are stem cells which have been matured such that they represent either a more differentiated state or a less differentiated state after contact with stimulatory factors.

### RELATED APPLICATIONS

The present application claims priority under 35 U.S.C. §119(e) to U.S. Provisional Patent Application No. 60/649,847 filed Feb. 2, 2005 and is a continuation-in-part of U.S. patent application Ser. No. 11/060,131 filed Feb. 16, 2005 which claims priority under 35 U.S.C. §119(e) to U.S. Provisional Patent Application No. 60/588,146 filed Jul. 15, 2004, the entire contents of which are incorporated herein by reference in their entirety.

### FIELD OF THE INVENTION

The present invention relates to the field of therapeutically reprogrammed cells. Specifically, therapeutically reprogrammed cells are provided that are not compromised by the aging process, are immunocompatible and will function in the appropriate post-natal cellular environment to yield functional cells after transplantation. More specifically, the present invention relates to therapeutic reprogramming cells with nuclear extracts that do not contain genetic material.

### BACKGROUND OF THE INVENTION

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types.

Stem cells are a rare population of cells that can give rise to vast range of cells tissue types necessary for organ maintenance and function. These cells are defined as undifferentiated cells that have two fundamental characteristics; (i) they have the capacity of self-renewal, (ii) they also have the ability to differentiate into one or more specialized cell types with mature phenotypes. There are three main groups of stem cells; (i) adult or somatic (post-natal), which exist in all post-natal organisms, (ii) embryonic, which can be derived from a pre-embryonic or embryonic developmental stage and (iii) fetal stem cells (pre-natal), which can be isolated from the developing fetus. Each group of stem cells has their own advantages and disadvantages for cellular regeneration therapy, specifically in their differentiation potential and ability to engraft and function de novo in the appropriate or targeted cellular environment.

In the post-natal animal there are cells that are lineage-committed progenitor stem cells and lineage-uncommitted pluripotent stem cells, which reside in connective tissues providing the post-natal organism the cells required for continual organ or organ system maintenance and repair. These cells are termed somatic or adult stem cells and can be quiescent or non-quiescent. Typically adult stem cells share two characteristics: (i) they can make identical copies of themselves for long periods of time (long-term self renewal); and (ii) they can give rise to mature cell types that have characteristic morphologies and specialized functions.

Much of the understanding of stem cell biology has been derived from hematopoietic stem cells and their behavior after bone marrow transplantation. There are several types of adult stem cells within the bone marrow niche, each having unique properties and variable differentiation ability in relation to their cellular environment. Somatic stem cells isolated from human bone marrow transferred in utero into pre-immune sheep fetuses have the ability to xenograft into multiple tissues. Also within the bone marrow niche are mesenchymal stem cells, which have a wide range of non-hematopoietic differentiation abilities, including bone, cartilage, adipose, tendon, lung, muscle, marrow stroma, and brain tissues. In addition, neural stem cells, pancreatic, muscle, adipose, ovarian and spermatogonial stem cells have been found. The therapeutic utility of somatic or post-natal stem cells has been demonstrated and realized through the use of bone marrow transplants. However, adult somatic stem cells have genomes that have been altered by aging and cell division. Aging results in an accumulation of free radical insults, or oxidative damage, that can predispose the cell to forming neoplasms, reduce cell differentiation ability or induce apoptosis. Repeated cell division is directly related to telomere shortening which is the ultimate cellular clock that determines a cells functional life-span. Consequently, adult somatic stem cells have genomes that have sufficiently diverged from the physiological prime state found in embryonic and prenatal stem cells.

Unfortunately, virtually every somatic cell in the adult animal's body, including stem cells, possess a genome ravaged by time and repeated cell division. Thus until now the only means for obtaining stem cells having an undamaged, or prime state physiological genome, was to recover stem cells from aborted embryos or embryos formed using in vitro fertilization techniques. However, scientific and ethical considerations have slowed the progress of stem cell research using embryonic stem cells. Generation of embryonic stem cell lines had been thought to provide a renewable source of embryonic stem cells for both research and therapy but recent reports indicate that existing cell lines have been contaminated with immunogenic animal molecules.

Another problem associated with using adult stems cells is that these cells are not immunologically privileged, or can lose their immunological privilege after transplant. (The term “immunologically privileged” is used to denote a state where the recipient's immune system does not recognize the cells as foreign). Thus, only autologous transplants are possible in most cases when adult stem cells are used. Thus, most presently envisioned forms of stem cell therapy are essentially customized medical procedures and therefore economic factors associated with such procedures limit their wide ranging potential. Additional barriers to the use of currently available

Moreover, stem cells must be induced to mature into the organ or cell type desired to be useful as therapeutics. The factors affecting stem cell maturation in vivo are poorly understood and even less well understood ex vivo. Thus, present maturation technology relies on serendipity and biological processes largely beyond the control of the administering scientist or recipient.

Current research is focused on developing embryonic stem cells as a source of totipotent or pluripotent immunologically privileged cells for use in cellular regenerative therapy. However, since embryonic stem cells themselves may not be appropriate for direct transplant as they form teratomas after transplant, they are proposed as “universal donor” cells that can be differentiated into customized pluripotent, multipotent or committed cells that are appropriate for transplant. Additionally there are moral and ethical issues associated with the isolation of embryonic stem cells from human embryos.

Therefore, there is a need for sources of biologically useful, pluripotent stem cells having genomes in a nearly physiologically prime state. Furthermore, there is a need for sources of biologically useful, pluripotent stem cells having genomes in a nearly physiologically prime state that maintain their immunological privilege in recipients for a time period sufficient to be therapeutically useful. Additionally, there is a need to condition stem cell transplants either in vivo or ex vivo in order to maximize the potential that the transplanted stem cell will mature into the intended tissue.

### SUMMARY OF THE INVENTION

The present invention provides biologically useful pluripotent therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications.

In one embodiment of the present invention, a method is provided for therapeutically reprogramming differentiated cells to yield pluripotent cells comprising isolating a differentiated cell, preparing a nuclear extract from a pluripotent stem cell; and incubating the differentiated cells with the nuclear extract to form reprogrammed pluripotent cells.

In another embodiment of the method of the present invention, the differentiated cell is any diploid (2N) cell derived from cells selected from the group consisting of a pre-embryonic, embryonic, fetal, and post-natal multi-cellular organisms or a primordial sex cell. In yet another embodiment, the pluripotent stem cell is selected from the group consisting of pre-embryonic stem cells, embryonic stem cells, fetal stem cells and post-natal stem cells.

In another embodiment of the method of the present invention, the nuclear extract does not contain genetic material, does not contain chromosomes or chromatin or contain DNA.

In an embodiment of the method of the present invention, the step of preparing a nuclear extract from a pluripotent stem cell comprises obtaining pluripotent stem cells, isolating karyoplasts from the pluripotent stem cells, removing the genetic material from the karyoplasts, and preparing an extract from the genetic-material deficient karyoplasts.

In another embodiment of the method of the present invention, the method further comprises the step of cryopreserving the reprogrammed pluripotent cells. In another embodiment, the method further comprises the step of transplanting the reprogrammed pluripotent cells into a patient in need thereof. In yet another embodiment, the reprogrammed pluripotent cell is autologous with the patient. In another embodiment the differentiated cell is in G₀.

In an embodiment of the present invention, a pluripotent cell useful for regenerative therapy in a patient in need thereof is provided comprising a differentiated cell therapeutically reprogrammed by exposure to a nuclear extract.

In another embodiment of the pluripotent cell of the present invention, the nuclear extract does not contain genetic material, does not contain chromosomes or chromatin or contain DNA.

In another embodiment of the pluripotent cell of the present invention, the differentiated cell is any diploid (2N) cell derived from cells selected from the group consisting of a pre-embryonic, embryonic, fetal, and post-natal multi-cellular organisms or a primordial sex cell. In yet another embodiment, the pluripotent stem cell is selected from the group consisting of pre-embryonic stem cells, embryonic stem cells, fetal stem cells and post-natal stem cells.

In another embodiment of the pluripotent cell of the present invention, the nuclear extract is prepared from a pluripotent stem cell.

In another embodiment of the pluripotent cell of the present invention, the pluripotent cell is cryopreserved. In yet another embodiment, the reprogrammed pluripotent cell is autologous with the patient.

### DEFINITION OF TERMS

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Embryo: As used herein, “embryo” refers to an animal in the early stages of growth and differentiation that are characterized implantation and gastrulation, where the three germ layers are defined and established and by differentiation of the germs layers into the respective organs and organ systems. The three germ layers are the endoderm, ectoderm and mesoderm.

Embryonic Stem Cell: As used herein, “embryonic stem cell” refers to any cell that is totipotent and derived from a developing embryo that has reached the developmental stage to have attached to the uterine wall. In this context embryonic stem cell and pre-embryonic stem cell are equivalent terms. Embryonic stem cell-like (ESC-like) cells are totipotent cells not directly isolated from an embryo. ESC-like cells can be derived from primordial sex cells that have been dedifferentiated in accordance with the teachings of the present invention.

Fetal Stem Cell: As used herein, “fetal stem cell” refers to a cell that is multipotent and derived from a developing multi-cellular fetus that is no longer in early or mid-stage organogenesis.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Multipotent: As used herein, “multipotent” refers to cells that can give rise to several other cell types, but those cell types are limited in number. An example of a multipotent cells is hematopoietic cells—blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Cell: As used herein, “somatic cell” refers to any cell in the body except gametes and their precursors.

Somatic Stem Cells: As used herein, “somatic stem cells” refers to diploid multipotent or pluripotent stem cells. Somatic stem cells are not totipotent stem cells.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

### DETAILED DESCRIPTION OF THE INVENTION

The present invention provides biologically useful pluripotent therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications.

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types.

The ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G₀/G₁ phase of the cell cycle. In mice and rats these gonocytes resume division within a few days after birth to generate spermatogonial stem cells and eventually undergo differentiation and meiosis related to spermatogenesis.

Primordial sex cells are directly responsible for generating the cells required for fertilization and eventually a new round of embryogenesis to create a new organism. Primordial sex cells are not programmed to die and are of a quality comparable to that of an embryonic state.

Embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage.

For the purpose of this discussion, an embryo and a fetus are distinguished based on the developmental stage in relation to organogenesis. The pre-embryonic stage refers to a period in which the pre-embryo is undergoing the initial stages of cleavage. Early embryogenesis is marked by implantation and gastrulation, wherein the three germ layers are defined and established. Late embryogenesis is defined by the differentiation of the germ layer derivatives into formation of respective organs and organ systems. The transition of embryo to fetus is defined by the development of most major organs and organ systems, followed by rapid fetal growth.

Embryogenesis is the developmental process wherein an oocyte fertilized by a sperm begins to divide and undergoes the first round of embryogenesis where cleavage and blastulation occur. During the second round, implantation, gastrulation and early organogenesis takes place. The third round is characterized by organogenesis and the last round of embryogenesis, wherein the embryo is no longer termed an embryo, but a fetus, is when fetal growth and development occurs.

During embryogenesis the first two tissue lineages arising from the morulae post-cleavage and compaction are the trophectoderm and the primitive endoderm, which make major contributions to the placenta and the extraembryonic yolk sac. Shortly after compaction and prior to implanting the epiblast or primitive ectoderm begins to develop.

The epiblast provides the cells that give rise to the embryo proper. Blastulation is complete upon the development of the epiblast stem cell niche wherein pluripotent cells are housed and directed to perform various developmental tasks during development, at which time the embryo emerges from the zona pellucida and implants to the uterine wall.

Implantation is followed by gastrulation and early organogenesis. By the end of the first round of organogenesis, all three germ layers will have been formed; ectoderm, mesoderm and definitive endoderm and basic body plan and organ primordia are established. Following early organogenesis, embryogenesis is marked by extensive organ development at which time completion marks the transformation of the developing embryo into a developing fetus which is characterized by fetal growth and a final round of organ development. Once embryogenesis is complete, the gestation period is ended by birth, at which time the organism has all the required organs, tissues and cellular niches to function normally and survive post-natally.

The process of embryogenesis is used to describe the global process of embryo development as it occurs, but on a cellular level embryogenesis can be described and/or demonstrated by cell maturation.

Fetal stem cells have been isolated from the fetal bone marrow (hematopoietic stem cells), fetal brain (neural stem cells) and amniotic fluid (pluripotent amniotic stem cells). In addition, stem cells have been described in both adult male and fetal tissues. Fetal stem cells serve multiple roles during the process of organogenesis and fetal development, and ultimately become part of the somatic stem cell reserve.

Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress.

There is a history of cellular therapy for the treatment of a variety of diseases but the majority of the use has been in bone marrow transplantation for hematopoietic disorders, including malignancies. In bone marrow transplantation, an individual's immune system is restored with the transplanted bone marrow from another individual. This restoration has long been attributed to the action of hematopoietic stem cells in the bone marrow.

There is increasing evidence that stem cells can be differentiated into particular cell types in vitro and shown to have the potential to be multipotent by engrafting into various tissues and transit across germ layers and as such have been the subject of much research for cellular therapy. As with conventional types of transplants, immune rejection is the limiting factor for cellular therapy. The recipient individual's phenotype and the phenotype of the donor will determine if a cell or organ transplant will be tolerated or rejected by the immune system.

Therefore, the present invention provides methods and compositions for providing functional immunocompatible stem cells for cellular regenerative/reparative therapy.

In an embodiment of the present invention, therapeutically reprogrammed cells are provided. Therapeutic reprogramming refers to a maturation process wherein a stem cell is exposed to stimulatory factors according the teachings of the present invention to yield pluripotent, multipotent or tissue-specific committed cells. The process of therapeutic reprogramming can be performed with a variety of stem cells including, but not limited to, therapeutically cloned cells, hybrid stem cells, embryonic stem cells, fetal stem cells, multipotent adult progenitor cells, adipose-derived stem cells (ADSC) and primordial sex cells.

Therapeutic reprogramming takes advantage of the fact that certain stem cells are relatively easily to obtain, such as spermatogonial stem cells and adipose-derived stem cells, and epigenetically reprograms these cells by exposure to stimulatory factors. These therapeutically reprogrammed cells have changed their maturation state to either a more committed cell lineage or a less committed cell lineage. Therapeutically reprogrammed cells are therefore capable of repairing or regenerating disease, damaged, defective or genetically impaired tissues.

Therapeutic reprogramming uses stimulatory factors including, without limitation, chemicals, biochemicals and cellular extracts to change the epigenetic programming of cells. These stimulatory factors induce, among other results, genomic methylation changes in the donor DNA. Quiescent spermatogonial stem cells (SSC) are particularly suitable for therapeutic reprogramming with the nuclear factors of the present invention. The quiescent SSCs are highly demethylated and therefore they are available for programming (patterning) or differentiation into any cell type.

Embodiments of the present invention include methods for preparing cellular extracts from whole cells, cytoplasts, nuclei and karyoplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation.

In one specific embodiment of the present invention, primordial sex cells (PSC) are therapeutically reprogrammed. Primordial sex cells, residing in the lining of the seminiferous tubules of the testes and the lining of the ovaries (the spermatogonia and oogonia, respectively) have been determined to possess diploid (2N) genomes remarkably undamaged by to the effects of aging and cell division. Thus, PSCs possess genomes in a nearly physiologically prime state. A non-limiting example of a PSC particularly useful in an embodiment of the present invention is a spermatogonial stem cell. According to the teachings herein, therapeutically reprogrammed PSC cells are prepared for the maturation process using means similar to that experienced by stem cells present in the developing embryo and fetus during embryogenesis and organogenesis.

Therapeutically reprogrammed cells made in accordance with the teachings of the present invention can be used for therapeutic purposes as is, they can be cryopreserved for future use or they can be further matured into a more committed cell lineage.

Embodiments of the present invention provide methods for further maturing or differentiating therapeutically reprogrammed cells, stem cells and primordial sex cells into more committed cell lineages in a post-natal environment to provide more committed cells for use in cellular regenerative/reparative therapy. In addition the maturation and differentiation process provides therapeutic cells that can be used to treat or replace damaged cells in pre- and post-natal organs.

The therapeutically reprogrammed cells made in accordance with the teachings of the present invention are useful in a wide range of therapeutic applications for cellular regenerative/reparative therapy. For example, and not intended as a limitation, the therapeutically reprogrammed cells of the present invention can be used to replenish stem cells in animals whose natural stem cells have been depleted due to age or ablation therapy such as cancer radiotherapy and chemotherapy. In another non-limiting example, the therapeutically reprogrammed cells of the present invention are useful in organ regeneration and tissue repair. In one embodiment of the present invention, therapeutically reprogrammed cells can be used to reinvigorate damaged muscle tissue including dystrophic muscles and muscles damaged by ischemic events such as myocardial infarcts. In another embodiment of the present invention, the therapeutically reprogrammed cells disclosed herein can be used to ameliorate scarring in animals, including humans, following a traumatic injury or surgery. In this embodiment, the therapeutically reprogrammed cells of the present invention are administered systemically, such as intravenously, and migrate to the site of the freshly traumatized tissue recruited by circulating cytokines secreted by the damaged cells. In another embodiment of the present invention, the therapeutically reprogrammed cells can be administered locally to a treatment site in need or repair or regeneration.

Stem cells are not universally susceptible to the maturation process of the present invention. Therefore the present inventors have developed a therapeutic reprogramming process whereby stem cells are induced into a state whereby they are susceptible to maturation factors. This therapeutic reprogramming process can be accomplished by incubation with stimulatory factors under suitable conditions and for a time sufficient to render the donor cell susceptible for maturation.

According to the teachings of the present invention, differentiated cells are reprogrammed by incubation with nuclear factors from pluripotent cells. The source of these nuclear factors can be any multipotent stem cell including, but is not limited to, multipotent stem cells isolated from pre-embryonic, embryonic, fetal or post-natal multicellular organisms. Nuclear factors for reprogramming are isolated from multipotent stem cell karyoplasts. In an embodiment of the present invention, the nuclear extracts contain the nuclear contents with genetic material removed. In yet another embodiment of the present invention the nuclear extracts lack intact or functional chromosomes. The scope of the present invention includes multipotent stem cell nuclear extracts having DNA and/or chromatin removed. Methods for preparing the nuclear extracts of the present invention are disclosed in Examples 3 and 4. Additional methods for removing genetic material from cells or karyoplasts include, but are not limited to, centrifugation, enzymatic treatment, precipitation, chromatography and other methods that are known to persons skilled in the art. The nuclear extracts made according to the teachings of the present invention can be cryopreserved after preparation.

Differentiated cells are treated with the nuclear extracts of the present invention by methods known to persons skilled in the art. Non-limiting examples of methods to treat differentiated cells with nuclear extracts include co-culture of differentiated cells with nuclear extracts and micro-injection of nuclear extracts into the nucleus of differentiated cells.

Pluripotentiality of extract-treated cells is determined by measuring the expression of the stem cell marker gene Oct4. Oct4 has an essential role in the control of developmental pluripotency and can activate or repress the expression of various genes. It is not known how Oct4 controls the pluripotent epigenotype but cells which have lost Oct4 expression are not plutipotent.

Nuclear extract-treated cells which express Oct4 are considered reprogrammed with a pluripotent epigenotype and are capable of differentiating into many cell types upon presentation with the appropriate differentiation factors in vitro or in vivo.

The following examples are meant to illustrate one or more embodiments of the invention and are not meant to limit the invention to that which is described below.

### EXAMPLE 1

### Isolation of Primordial Sex Cells from Testes

The testes were excised and decapsulated. Testicular tissue was minced using fine scissors and transferred into culture medium (DMEM/F12) containing 1 mg/mL collagenase type I (Sigma) and 0.5 mg/mL DNase (Sigma). Digestion was performed at 37° C. for 10 min in a shaking water bath operated at 110 cycles/min. Interstitial cells are separated by sedimentation at unit gravity for 10 min and washed in DMEM/F12.

A final digestion of the basal lamina components of the testicular tissue was carried out in a mixture of collagenase type I (1 mg/mL), DNase (0.5 mg/mL), and hyaluronidase (Sigma; 0.5 mg/mL) under the same conditions as for the first digestion step. The single-cell suspension obtained was washed successively with medium and PBS containing 1 mM EDTA (Sigma) and 0.5% fetal calf serum. The undigested remains of the tunica albuginea were eliminated by filtering the cell suspension through a 50 μm nylon mesh. All cells were kept at 5° C. throughout the procedure. The dissociated testicular cells were suspended (5×10⁶ cells/mL) in PBS containing 0.5% FBS (PBS/FBS). The cells were then incubated with primary antibodies for 20 min on ice, washed twice with excess PBS/FBS, and used for FACS analysis. Primary antibodies include R-phycoerythrin (PE)-conjugated anti-α6-integrin, allophycocyanin (APC)-conjugated anti-c-kit, and biotinylated anti-αv-integrin. For experiments using secondary reagents, cells were further incubated for 20 min with APC-conjugated streptavidin to detect biotinylated antibody. All antibodies or secondary reagents were used at 5 μg/mL. Control cells were not treated with antibodies. After the final wash, the cells were resuspended (10⁷ cells/mL) in 2 mL PBS/FBS containing 1 μg/mL propidium iodide (Sigma), filtered into a tube through a 35 μm pore-size nylon screen, and kept in the dark on ice until analysis. The cells were sorted based on antibody staining and their relative granularity or internal complexity (side scatter, SSC). Cell sorting was performed by a dual-laser FACStar Plus (Becton Dickinson) equipped with 488-nm argon (200 mW) and 633-nm helium neon (35 mW) laser. An argon laser was used to excite PE and propidium iodide, and emissions were collected with a 575 DF 26 filter for PE and a 610 DF 20 filter for propidium iodide. A neon laser was used to excite APC, and emission was detected with a 675 DF 20 filter. Dead cells were excluded by eliminating propidium iodide-positive events at the time of data collection. Cells were sorted into 5 mL polystyrene tubes containing 2 mL of ice-cold DMEM supplemented with 10% FBS (DMEM/FBS). The α6-integrin^{hi}/SSC^{lo}/c-kit(-) population was used as the donor cell.

### EXAMPLE 2

### Isolation of Primordial Sex Cells from Ovaries

The animal is anesthetized and the ovaries are removed. Alternatively, primordial sex cells (PSCs) can be isolated from a punch biopsy of the ovaries. The PSCs are then isolated with the assistance of a microscope. Primordial sex cells have stem cell morphology (i.e. large, round and smooth) and are mechanically retrieved from the ovaries.

### EXAMPLE 3

### Therapeutic Reprogramming with Karyoplast Extracts

This example describes the therapeutic reprogramming of a PSC so that it is functional and responds appropriately during maturation by inducing genomic modifications using nuclear (karyoplast) extracts from embryonic stem cells.

Primordial sex cells were isolated as described in Examples 1 and 2. The α6-integrin^{hi}/SSC^{lo}/c-kit(-) population is used as the reprogrammable cell. These cells were stored on ice until exposure to nuclear extracts.

For preparation of embryonic stem cell nuclear (karyoplast) extracts, the embryonic stem cells (ESCs) are cultured to confluency. The ESC karyoplasts are prepared using a discontinuous density gradient of Ficoll-400 (30%, 25%, 22%, 18% and 15%) containing 10 μg/mL cytochalasin B. Ten million ESCs in 12.5% Ficoll-400 are carefully layered on top of the gradient and centrifuged at 40,000 rpm at 36° C. for 30 min. The karyoplasts are collected from the 30% level. The karyoplasts are then washed three times with ice-cold PBS followed by a wash in cell lysis buffer. The karyoplasts are then centrifuged at 350×g and resuspended in 1.5 volumes of cell lysis buffer containing protease inhibitors and incubated on ice for 45 min. The karyoplasts are then homogenized by pulse sonication and then the karyoplasts are centrifuged at 16,000×g for 20 min at 4° C. The supernatant is then collected and protein concentration determined to be approximately 6 mg/mL.

The previously isolated PSCs are washed three times with ice-cold PBS, followed by a two washes in HBSS. The cells are then centrifuged at 350×g for 5 min at 4° C. and resuspended at 10,000 cells per 14 μL of ice-cold HBSS. The cells are then incubated at 37° C. for 2 min followed by the addition of streptolysin O (SLO; Sigma) at a final concentration of 115 ng/mL to 230 ng/mL depending on cell number and incubated for 50 min at 37° C. with constant shaking to keep the cells from sedimenting. The cells are then centrifuged at 500×g for 5 min at 4° C. and the supernatant removed. The PSCs are then incubated with 50 μL of previously prepared embryonic stem cell extract containing an ATP-regenerating system and 1 mM of each of the four nucleoside triphosphates (NTP) at 37° C. for 1-2 hours. The cells are then resuspended in solution of 2 mM CaCl2 in preparation media (1% nonessential amino acids, 1% L-glutamine, 100 units/mL penicillin, 100 μg/mL streptomycin, 0.1 mM β-mercaptoethanol, 3,000 units/mL of leukemia inhibitory factor (LIF) in DMEM/20% FBS) and placed into one well of a 48-well dish pre-treated with 0.1% gelatin containing a mitomycin C-inactivated primary embryonic fibroblast (PEF) layer. In addition, it is also possible to co-culture the extract-treated PSCs in a 48-well dish pre-treated with 0.1% gelatin containing a mitomycin C-inactivated PEF layer and 50% confluent ESCs. After 24 hours, cells that were not attached to the feeder layer were removed and the extract exposure procedure was repeated a second time with the unattached cells. The reprogrammed cells (attached cells) are cultured and assayed for embryonic stem cell specific markers (i.e. REX1, OCT4). Additionally the extract-treated (reprogrammed) cells can be tested for in vitro differentiation potential prior to being exposed to a maturation process.

### EXAMPLE 4

### Nuclear Extract Preparation

For preparation of stem cell nuclear extracts, the stem cells are plated two days prior to extract preparation and allowed to reach maximum monolayer growth in preparation media on 25×75 mm tissue culture slides pre-treated with 10 ng/mL fibronectin at 37° C. for 1 hour. On the day of extract preparation, 2 μg/mL of cytochalasin D (final concentration) is added to the media and the slides incubated for 120 min at 37° C. Following the 120 min incubation step, the slides are centrifuged in a swinging bucket centrifuge at 10,000×g for 1 hour in preparation media containing 2 μg/mL of cytochalasin D. Prior to centrifugation, the rotor and centrifuge are pre-warmed to 37° C. After centrifugation, the pellet containing the nuclei is washed three times with ice-cold PBS, followed by one wash in cell lysis buffer. The cells are then centrifuged at 350×g and resuspended in 1.5 volumes of cell lysis buffer containing protease inhibitors and the nuclei are incubated on ice for 15-45 min. The nuclei are homogenized by pulse sonication. The lysates are then centrifuged at 16,000×g for 20 min at 4° C. The supernatant is then collected and protein concentration determined.

### EXAMPLE 5

### Incubation of Cells with Nuclear Extracts

The previously isolated stem or somatic cells are washed three times with ice-cold PBS, followed by a two washes in HBSS. The cells are then centrifuged at 350×g for 5 min at 4° C. and resuspended at 10,000 cells per 14 μL of ice-cold HBSS. The cells are then incubated at 37° C. for 2 min followed by the addition of streptolysin O at a final concentration of 115 ng/mL to 230 ng/mL depending on cell number and incubated for 50 min at 37° C. with constant shaking to keep the cells from sedimenting. The cells are then centrifuged at 500×g for 5 min at 4° C. and the supernatant removed. The stem or somatic cells are then incubated with 50 μL of previously prepared stem cell nuclear extracts containing an ATP-regenerating system and 1 mM of each of the four NTPs at 37° C. for 1-2 hours. The cells are then resuspended in solution of 2 mM CaCl₂ in preparation media (1% nonessential amino acids, 1% L-glutamine, 100 units/mL penicillin, 100 μg/mL streptomycin, 0.1 mM β-mercaptoethanol, 3,000 units/mL of LIF in DMEM/20% FBS) and placed into one well of a 48-well dish pre-treated with 0.1% gelatin containing a mitomycin C-inactivated primary embryonic fibroblast layer. After 24 hours, cells that were not attached to the feeder layer were removed and the extract exposure procedure was repeated a second time with the unattached cells. The reprogrammed cells (attached cells) are cultured are then available for transplant directly into a recipient, cryopreserved for future use, subjected to a maturation process or fused with appropriate host cells to generate hybrid stem cells.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for therapeutically reprogramming differentiated cells to yield pluripotent cells comprising:
isolating a differentiated cell;
preparing a nuclear extract from a pluripotent stem cell; and
incubating said differentiated cells with said nuclear extract to form reprogrammed pluripotent cells.

2. The method of claim 1 wherein said differentiated cell is any diploid (2N) cell derived from cells selected from the group consisting of a pre-embryonic, embryonic, fetal, and post-natal multi-cellular organisms or a primordial sex cell.

3. The method of claim 1 wherein said pluripotent stem cell is selected from the group consisting of pre-embryonic stem cells, embryonic stem cells, fetal stem cells and post-natal stem cells.

4. The method of claim 3 wherein said pluripotent stem cell comprises an embryonic stem cell.

5. The pluripotent cell of claim 1 wherein said nuclear extract does not contain genetic material.

6. The pluripotent cell of claim 1 wherein said nuclear extract does not contain chromosomes or chromatin.

7. The pluripotent cell of claim 1 wherein said nuclear extract does not contain DNA.

8. The method of claim 1 wherein said preparing step comprises:
obtaining pluripotent stem cells;
isolating karyoplasts from said pluripotent stem cells;
removing the genetic material from said karyoplasts; and
preparing an extract from said genetic-material deficient karyoplasts.

9. The method of claim 1 further comprising the step of cryopreserving said reprogrammed pluripotent cells.

10. The method of claim 1 further comprising the step of transplanting said reprogrammed pluripotent cells into a patient in need thereof.

11. The method of claim 10 wherein said reprogrammed pluripotent cell is autologous with said patient.

12. The method of claim 1 wherein said differentiated cell is in G₀.

13. A pluripotent cell useful for regenerative therapy in a patient in need thereof comprising:
a differentiated cell therapeutically reprogrammed by exposure to a nuclear extract.

14. The pluripotent cell of claim 13 wherein said nuclear extract does not contain genetic material.

15. The pluripotent cell of claim 13 wherein said nuclear extract does not contain chromosomes or chromatin.

16. The pluripotent cell of claim 13 wherein said nuclear extract does not contain DNA.

17. The pluripotent cell of claim 13 wherein said differentiated cell is any diploid (2N) cell derived from cells selected from the group consisting of a pre-embryonic, embryonic, fetal, and post-natal multi-cellular organisms or a primordial sex cell.

18. The pluripotent cell of claim 13 wherein said nuclear extract is prepared from a pluripotent stem cell.

19. The pluripotent cell of claim 13 wherein said pluripotent cell is cryopreserved.

20. The pluripotent cell of claim 13 wherein said differentiated cell is autologous with said patient.
